(19) United States
(12) Patent Application Publication
(10) Pub. No.: US 2002/0187543 A1
(43) Pub. Date: Dec. 12, 2002

(54) Enhanced dispersion of adenoviral vectors by fusogenic membrane glycoproteins

(76) Inventors: David T. Curiel, Birmingham, AL (US); Jerry L. Blackwell, Birmingham, AL (US)

Correspondence Address: Benjamin Aaron Adler, ADLER & ASSOCIATES, 8011 Candle Lane, Houston, TX, 77071, US

(21) Appl. No.: 10/117,488

(22) Filed: Apr. 5, 2002

Related U.S. Application Data
(60) Provisional application No. 60/281,847, filed on Apr. 5, 2001.

(51) Int. Cl. A61K 48/00; C12N 7/00; C12N 15/861

(52) U.S. Cl. 435/235.1; 424/93.2; 435/456

(57) ABSTRACT

The present invention provides a method of increasing adenoviral vector dispersion within tumor mass by inducing syncytia formation with fusogenic membrane glycoproteins. These data demonstrate the utility of fusogenic membrane glycoproteins as dispersion agents and suggest that fusogenic membrane glycoproteins can improve the efficacy of conditionally replicative adenovirus gene therapy.

### CROSS-REFERENCE TO RELATED APPLICATION

This non-provisional patent application claims benefit of provisional patent application U.S. Ser. No. 60/281,847, filed Apr. 5, 2001, now abandoned.

### FEDERAL FUNDING LEGEND

This invention was produced in part using funds obtained through a grant from the National Institutes of Health. Consequently, the federal government has certain rights in this invention.

### BACKGROUND OF THE INVENTION

1. Field of the Invention

The present invention relates generally to the field of adenoviral vectors. More specifically, the present invention relates to the use of viral fusogenic membrane glycoproteins to enhance dispersion of adenoviral vectors.

2. Description of the Related Art

Advancements in modern medicine have led to improved treatments for neoplastic diseases. However, a number of malignancies are still refractory to conventional medical interventions such as chemotherapy, radiation therapy, and surgery. This drawback to conventional medical interventions supports the development of new therapeutic agents that can either augment the effects of these conventional therapies or serve as alternative treatments.

One such promising new treatment modality is adenovirus (Ad)-mediated gene therapy, which offers a broad range of interventions such as mutation compensation, molecular chemotherapy, and antitumor immune stimulation (Zhang, 1999). In this regard, many preclinical adenovirus-mediated cancer gene therapy studies have yielded promising results suggesting their successful translation into clinical applications. However, adenovirus-mediated gene transfer has frequently demonstrated limited efficacy in vivo.

Attempts to overcome this problem have included the development of new adenoviral vectors with enhanced transduction efficiency (Wickham et al., 1997; Krasnykh et al., 1998; Kasono et al., 1999; Reynolds et al., 1999; Vanderkwaak et al., 1999; Staba et al., 2000; Suzuki et al., 2001). To further improve on adenoviral vector performance, conditionally replicative adenoviruses (CRAds) have been designed to facilitate tumor-specific oncolytic replication (Bischoff et al., 1996; Dachs et al., 1997; Rodriguez et al., 1997; Alemany et al., 2000; Curiel, 2000; Doronin et al., 2001; Suzuki et al., 2001; Yu et al., 2001).

To date, tumor-specific replication has been based on two major strategies. In the first strategy, the native promoters for adenoviral genes have been replaced with tumor-specific promoters, such as those from the prostate-specific antigen (Rodriguez et al., 1997), kallikrein 2 (Yu et al., 1999b), rat probasin (Yu et al., 1999a) or α-fetoprotein (Hallenbeck et al., 1999) promoters. The second strategy involves the removal of viral functions that are dispensable in tumor cells, such as the genes responsible for activating the cell cycle through p53 (Bischoff et al., 1996; Ganly et al., 2000) or retinoblastoma protein (Rb) binding (Fueyo et al., 2000). As more information becomes available regarding tumor molecular biology and adenovirus-host cell interactions, additional molecular targets for conditionally replicative adenoviruses will undoubtedly emerge.

For conditionally replicative adenoviral vectors to be more effective, they must disperse efficiently throughout the tumor mass (Kirn, 2000; Sauthoff et al., 2000). The ability of the virus to lyse target cells and infect new cells correlates directly with efficacy of the virotherapy (Alemany et al., 2000; Sauthoff et al., 2000). The prior art is deficient in methods of enhancing dispersion of adenoviral vectors in tumor tissue. The present invention fulfills this long-standing need and desire in the art by employing the natural cellular membrane fusion activity of viral fusogenic membrane glycoproteins.

### SUMMARY OF THE INVENTION

Conditionally replicative adenovirus (CRAd) vectors are designed for specific oncolytic replication in tumor tissues with concomitant sparing of normal cells. As such, conditionally replicative adenoviruses offer an unprecedented level of anticancer potential for malignancies that have been refractory to previous cancer gene therapy interventions. Conditionally replicative adenoviruses efficacy may, however, be compromised by inefficient dispersion of the replicating vector within the tumor tissue.

The present invention evaluates the utility of fusogenic membrane glycoprotein (FMG), which induces the fusion of neighboring cellular membranes to form multinucleated syncytia, in enhancing the dispersion of viral vector throughout the tumor mass. Human immunodeficiency virus type 1 (HIV-1) envelope glycoproteins, which induce syncytia in the presence of CD4⁺ target cells, were expressed by an Ad (Ad5HIVenv) in permissive (CD4-positive) and nonpermissive (CD4-negative) cell lines. The results demonstrated that syncytium formation was compatible with adenoviral replication and dramatically increased the dispersion of virus gene products within the cytoplasm of the syncytia as well as viral particles in the nuclei of the syncytial mass. Moreover, progeny virions were released more efficiently from syncytia compared with nonsyncytial cells. These data demonstrate the utility of FMGs as a dispersion agent and suggest that FMGs can improve the efficacy of conditionally replicative adenovirus gene therapy.

The present invention is drawn to a method of increasing adenoviral vector dispersion within a tumor mass by infecting the tumor mass with an adenoviral vector expressing viral fusogenic membrane glycoproteins that induce syncytia formation in the tumor mass. In one embodiment, the cancer cells are of cervical cancer origin and the fusogenic membrane glycoprotein is from the human immunodeficiency type-1 virus (HIV-1).

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 shows detection of syncytia in the HIV-1 FMG and HeLa/HeLa-CD4 1 model system. HeLa (a-c) and HeLa-CD4 1 (d-f) cells were mock infected (a and d) or infected with Ad5HIVenv (b, c, e, and f) at a multiplicity of infection of 10 PFU/cell. Approximately 24 hr later, the monolayers were visualized by bright-field microscopy at an original magnification of ×10 (a, b, d, e) and ×20 (c and f). Pseudocolors were applied to the digital images to enhance the contrast of the cytoplasm (blue) and nuclei (yellow-green).

FIG. 2 shows validation of the cell components of the syncytia model. FIG. 2A shows flow cytometric analysis of the primary adenovirus receptor, CAR, in the HeLa and HeLa-CD4⁺ cells. Cells were labeled with an isotype control IgG (M1) or the anti-CAR antibody RmcB (M2) and then an FITC-conjugated anti-mouse IgG antibody. FIG. 2B shows triplicate wells of HeLa and HeLa-CD4⁺ cells infected with equal amounts (0.1, 1.0, or 10 MOI) of a luciferase-expressing adenovirus. Approximately 24 hr later the cells were lysed and relative luciferase units (RLU) was measured.

FIG. 3 shows comparison of adenovirus transgene expression in the presence and absence of syncytia. FIG. 3A shows dispersion of adenovirus transgene products in syncytia. HeLa (a and b) and HeLa-CD4⁺ (c and d) cells were coinfected with Ad5HIVenv (10 MOI) and Ad5GFP (10 MOI). Approximately 24 hr later, the monolayers were visualized by fluorescence microscopy at an original magnification of ×10 (a and c) and ×20 (b and d). FIG. 3B shows quantification of transgene expression. Quadruplicate wells of HeLa and HeLa-CD4⁺ cells were coinfected with Ad5HIVenv (10 MOI) and Ad5Luc1 (0.1, 1.0, or 10 MOI). Approximately 24 hr later, the luciferase activity was measured.

FIG. 4 shows analyses of de novo virus production. FIG. 4A shows HeLa and HeLa-CD4 1 cells were infected with Ad5HIVenv (MOI of 10) and then the amounts of virus in the cell and medium fractions at 24, 48, 72, and 96 hr after infection were determined by plaque assays on 293 cells. The amounts of virus in the cell and medium fractions were added together to give the net virus yield. FIG. 4B shows the amounts of virus in the medium or cell fraction at each time point. The point at which the amount of free virus (medium fraction) exceeds the amount of cell-associated virus, or burst point (arrow), in the infected HeLa-CD4 1 cells is indicated. FIG. 4C shows the ratio of free to total virus (titer of medium fraction divided by the sum of the titer of cell fraction plus the medium fraction).

FIG. 5 shows high-resolution analysis of FMG-induced syncytium formation and virus particle dispersion. HeLa and HeLa-CD4⁺ cells were infected with Ad5HIVenv and 30 hr later the monolayers were visualized by electron microscopy. FIG. 5 A shows an infected HeLa cell (nucleus number 1) with a clear interstitial boundary (arrows). Similar boundaries delineate both infected and uninfected cells. Original magnification: ×3000. FIG. 5B shows a field of syncytia with no apparent interstitial boundaries between cells or nuclei. Original magnification: ×2000. FIG. 5C shows nuclei 1 and 2 [same as in (B)] were positive for virus particles. Nuclei 3-7 and 9 had detectable amounts of virus particles as well. Original magnification: ×2,000,000.

FIG. 6 shows syncytia in prostate cancer cells using the Mason-Pfizer monkey virus fusogenic membrane glycoprotein. Prostate cancer cells were transfected with an irrelevant plasmid that expresses the luciferase reporter gene (panel a) or with pMPMVenv that expresses the Mason-Pfizer monkey virus fusogenic membrane glycoprotein (panel b). Panel b shows the characteristic morphological changes that occur when cell-to-cell fusion is induced by the fusogenic membrane glycoproteins.

FIG. 7 shows a proposed mechanism of viral dispersion using fusogenic membrane glycoprotein-induced cell-to-cell fusion. During the infection phase, the FMG-expressing Ad infects a subpopulation (1-10% of total) of target tumor cells. Subsequently, viral genes (X) and FMGs (˜) are expressed during the transgene expression phase. The expressed FMGs cause nearby cells to fuse with the originally infected cell. Also during the early syncytia phase, the viral gene products are dispersed throughout the syncytial mass. As Ad replication cycle continues into the intermediate syncytia phase, virus particles assemble and disperse throughout the syncytial mass.

### DETAILED DESCRIPTION OF THE INVENTION

Various drugs, as well as physical and enzymatic treatments, have been evaluated as a means to increase the spread of the virus throughout the tumor mass (Jain, 1990a,b; Kuriyama et al., 2000; Nevo et al., 2001). Employing the natural cellular membrane fusion activity of viral fusogenic membrane glycoproteins (FMGs) is another possible method to accomplish virus dispersion. In this regard, human immunodeficiency virus type 1 (HIV-1) is one such virus that possesses inherent fusogenic activity. HIV-1 binds the CD4 molecule, normally expressed on a subset of T lymphocytes, monocyte-macrophages, and dendritic cells, as one of the earliest steps of viral entry into the host cell (Sattentau and Moore, 1993). HIV-1 requires, in addition to CD4, a chemokine receptor (CXCR4 or CCR5) for viral entry to occur (reviewed in Hoffman and Doms, 1998; Littman, 1998).

Entry of HIV-1 into the target cell is mediated by two Env glycoproteins, gp120 and gp41, that are proteolytically processed from a precursor molecule, gp160 (Luciw, 1996; Hunter, 1997). The surface glycoprotein, gp120, mediates CD4 and coreceptor binding (Kwong et al., 1998) and the viral transmembrane glycoprotein, gp41, anchors the glycoprotein complex within the host-derived viral membrane and mediates virus-cell membrane fusion. Analogous to the HIV-1 entry mechanism, cellular membrane fusion can also occur when cells expressing CD4 and an appropriate chemokine receptor come into contact with cells expressing HIV-1 Env. The sequential fusion of cells by this mechanism results in the formation of large syncytia.

In the context of conditionally replicative adenoviruses—mediated gene therapy, these fusogenic membrane glycoprotein-induced syncytia may facilitate the transfer of Ad gene products and viral particles to uninfected cells via fusion of an infected tumor cell with other uninfected tumor cells, a mechanism that is analogous to that of measles virus replication (Duprex et al., 1999; Firsching et al., 1999). Historically, fusogenic membrane glycoproteins were expressed from their native viruses such as influenza virus (Huang, 1985), measles virus (Schattner et al., 1985), vesicular stomatitis virus (Moller et al., 1985; Schattner et al., 1985), and Newcastle disease virus (Reichard et al., 1992; Lorence et al., 1994). These fusogenic viruses were used therapeutically to induce oncolysis and elicit potent anti-tumor immune responses (see review in Sinkovics and Horvath, 1993). In the case of Newcastle disease virus therapy, tumor cell death was preceded by virus-induced syncytium formation (Sinkovics and Horvath, 1993).

More recently, recombinant DNA forms of fusogenic membrane glycoproteins have been expressed from plasmid and heterologous viral vectors (Bateman et al., 2000; Diaz et al., 2000; Fielding et al., 2000; Higuchi et al., 2000; Galanis et al., 2001). Importantly, expression of the recombinant protein from these vectors has been shown to retain the biological activity of the fusogenic membrane glycoprotein, which has prompted a resurgence of interest in fusogenic membrane glycoproteins as antitumor agents. As immunological antitumor agents, fusogenic membrane glycoproteins have been shown to induce expression of at least two different heat shock proteins (Bateman et al., 2000) whose activation is known to enhance tumor immunogenicity (Tamura et al., 1997; Melcher et al., 1998). In addition, induction of syncytium formation has been shown to have a direct killing effect on the target cells (Bateman et al., 2000; Diaz et al., 2000; Higuchi et al., 2000; Galanis et al., 2001; Haviv et al., 2001). Collectively, the predicted dispersion activities and reported therapeutic effects make fusogenic membrane glycoproteins attractive moieties for novel cancer gene therapy applications.

Although fusogenic membrane glycoproteins have been convincingly described as extremely potent anticancer agents (Bateman et al., 2000; Diaz et al., 2000; Higuchi et al., 2000), it is not evident whether the induction of syncytium formation is biologically compatible with a replicating adenoviral system. This is an important question to address since it is reasonable to predict that fusogenic membrane glycoprotein expression and subsequent syncytium formation may be deleterious to efficient adneoviral gene expression and productive infection. Previous studies have used either nonviral vectors (Bateman et al., 2000; Fielding et al., 2000) or other viral vector systems (Diaz et al., 2000) to study the utility of fusogenic membrane glycoproteins-based therapies. The noted disadvantages of these vector systems are their inherent low gene transfer efficiencies and/or inability to produce high-titer virus preparations.

The present invention demonstrates that HIV-1 FMG-induced syncytia are compatible with a replicating adenovirus. At early and intermediate phases of syncytium formation (≦24 hr postinfection), adenovirus gene expression was largely unaffected b y syncytium formation. During the first ˜72 hr postinfection, virus particle production was actually higher in the presence of syncytia, ranging from 0.5 to 1.0 log more net virus. It is not known how syncytium formation may have stimulated virus progeny production; however, a few reports have shown that HIV-1 Env proteins can modulate host gene expression (Merzouki et al., 1995) and signal transduction pathways (Westendorp et al., 1995; Wyss-Coray et al., 1996). Despite the uncertainties of the mechanism(s), the biocompatibility of fusogenic membrane glycoprotein-induced syncytia with Ad replication further validates the use of fusogenic membrane glycoproteins as an anticancer gene therapy modality.

In addition to being compatible with adenoviral replication, the present invention shows that fusogenic membrane glycoprotein-induced syncytium formation has a profound effect on the distribution of adenoviral gene products and viral progeny. Rather than being isolated to individual cells in the absence of syncytia, adenoviral gene products are dispersed throughout the syncytial mass. Neither a net gain nor loss of transgene activity was associated with this redistribution. However, there was a net gain in the number of cells exposed to the transgene products in the presence of syncytia. This effect may prove to be particularly interesting in the case of enzyme/prodrug-based therapies, since more of the pro-drug-converting enzyme and the toxic drug will be able to reach a larger number of target cells. An extensive redistribution of new virus particles in the syncytial masses was also observed. Interestingly, in many instances a gradient of virus particle numbers per nuclei in a given syncytium existed. This observation supports the idea that one originally infected cell initiated the formation of the syncytium, followed by the subsequent involvement of other neighboring cells. As more cells became incorporated into syncytia, there was a dispersion of virus particles, resulting in a more pervasive infection of the tumor mass.

In addition to their dispersion within the syncytium, progeny virus particles were released more efficiently from cells in syncytia. Importantly, this faster release of the virus did not have a negative influence on the amount of total functional virus produced. The higher “burst rate” would predict more efficient secondary infection of cells that were not initially infected and were not incorporated into a syncytial mass. In summary, this novel dispersion mechanism improves replicating adenovirus-based gene therapy on three levels: (1) lateral spread of viral and/or therapeutic genes, (2) dispersion of viral particles to cells that were not initially infected, and (3) more rapid release of virus progeny for subsequent infection cycles. Collectively, these data demonstrate how the activity of fusogenic membrane glycoprotein can be exploited to augment an indispensable constituent of effective replicating adenoviral gene therapy—spread of the infection.

Whereas this study intentionally focused on early and intermediate stages of syncytium formation as a mechanism to disperse virus particles and gene products, the utility of fusogenic membrane glycoprotein-induced syncytium formation as a direct anticancer modality has also been established (Sinkovics and Horvath, 1993; Melcher et al., 1998; Bateman et al., 2000; Diaz et al., 2000; Higuchi et al., 2000; Galanis et al., 2001). This late-stage syncytial event has been previously shown in in vivo treatment models using relatively small tumor nodules. Using the adenovirus vector to deliver the fusogenic membrane glycoprotein, preliminary data suggest an ability to treat much larger established tumors in mouse xenograph models. There was also a noted concern that the more rapid release of virus progeny from tumor cells may result in significant systemic toxicity. Experiments using the mouse xenograph model have indicated that no additional toxicity results from the efficient release of virus progeny in the tumor extracellular milieu.

The HIV-1 fusogenic membrane glycoprotein-based model disclosed herein establishes the utility of fusogenic membrane glycoproteins as antitumor agents. In addition, fusogenic membrane glycoproteins with a broader tropism of syncytium-inducing activity may be employed. For example, the Mason-Pfizer monkey virus Env protein (Ogura et al., 1978; Brody and Hunter, 1992) can be used to induce syncytium formation in prostate, breast, and head and neck cancer cells. Syncytin (Mi et al., 2000), a human endogenous retrovirus fusogenic membrane glycoprotein that directs syncytium formation in placental syncytiotrophoblasts, is another candidate fusogenic membrane glycoprotein that induces syncytium formation in CD4-negative cells (An et al., 2001).

Central to the utility of fusogenic membrane glycoprotein-based therapies is the capacity to restrict syncytium formation to target cancer cells and tissues. In this regard, matrix metalloproteinase cleavable linkers (Johnson et al., 2001) and an inducible rapamycin transcriptional control system (Chong et al., 2001) can be used to regulate fusogenic membrane glycoprotein expression and cytotoxicity. In addition, placing the fusogenic membrane glycoprotein gene under the control of a tumor-specific promoter or expressing the fusogenic membrane glycoprotein gene in the context of a CRAd would restrict syncytium formation to target cancer tissues. In conclusion, the development of a highly regulated expression system using permissive fusogenic proteins that interact with a larger repertoire of target cells will expand this approach to a broader spectrum of malignancies.

As used herein, “fusogenic membrane glycoprotein” refers to any of the type of proteins that cause fusion of cell membranes to form a multinucleated syncytia.

The present invention is directed to a method of increasing adenoviral vector dispersion within tumor mass b y infecting the tumor mass with an adenoviral vector expressing a viral fusogenic membrane glycoprotein and inducing syncytia formation by the fusogenic protein. Representative viral fusogenic membrane glycoproteins include, but are not limited to, respiratory syncytia virus, Mason-Pfizer monkey virus, measles virus, human endogenous retrovirus. Generally, this method may be used to treat a tumor or cancer such as cervical cancer, prostate cancer, squamous cell carcinoma of the head and neck, breast cancer, and lung cancer. In a preferred embodiment, the tumor is cervical cancer and the fusogenic membrane glycoprotein is from HIV-1.

The following examples are given for the purpose of illustrating various embodiments of the invention and are not meant to limit the present invention in any fashion.

### EXAMPLE 1

### Cells And Viruses

HeLa and HeLa-CD4⁺ (clone HI-Q) cell lines were obtained from D. Kabat (Kabat et al., 1994). The HeLa-CD4⁺ cell line was derived from HeLa cells and was modified to constitutively express the CD4 receptor. Both HeLa and HeLa-CD4⁺ cell lines endogenously express the HIV-1 coreceptor CXCR4. Cell lines were maintained in Eagle's minimum essential medium with 2 mM L-glutamine and Earle's balanced salt solution (BSS) with sodium bicarbonate (1.5 g/liter), 0.1 mM nonessential amino acids, 1.0 mM sodium pyruvate, and 10% fetal bovine serum at 37° C. in 5% CO2. The HIV-1 envelope-expressing adenovirus, Ad5HIVenv, was generously provided by V. Natarajan (Dewar et al., 1989). Ad5Luc and Ad5GFP, which express the luciferase and green fluorescent protein reporter genes respectively, were generated at the University of Alabama (UAB, Birmingham, Ala.) Gene Therapy Center. Viruses were propagated and plaque titered (Graham and van der Eb, 1973) on 293 cells and purified by double centrifugation on cesium chloride gradients as described (Graham and Prevec, 1991). The physical titers were determined spectrophotometrically by measuring the optical density (OD) at 260 nm, where 1 absorbance unit is equivalent to 1.1×10¹² vector particles (Maizel et al., 1968).

### EXAMPLE 2

### Flow Cytometry

Cell monolayers were washed once with phosphate-buffered saline (PBS) (without Mg²⁺ and Ca²⁺) and then incubated with Versene (GIBCO-BRL, Gaithersburg, Md.) at 37° C. in a CO₂ incubator until the cells were released from the plate surface. After centrifugation at 400×g for 5 min, the cell pellet was resuspended into PBS containing 1% bovine serum albumin (BSA) and 0.1% sodium azide (PBS-BSA-azide). Approximately 2.0×10⁵ cells in 1.0 ml of PBS-BSA-azide were incubated with the anti-CAR (coxsackievirus-adenovirus receptor) antibody (2.0 μg/ml), RmcB (Hsu et al., 1988), or normal mouse IgG (2.0 μg/ml) for 1 hr at 4° C. Cells were then washed three times with PBS-BSA-azide and incubated with fluorescein isothiocyanate (FITC)-conjugated anti-mouse IgG antibody (Sigma, St. Louis, Mo.) at a concentration of 0.1 μg/ml for 1 hr at 4° C. Cells were washed once with PBS-BSA-azide and twice with PBS-azide and then analyzed by flow cytometry at the Immunopathology Program Flow Cytometry Facility (University of Alabama at Birmingham).

### EXAMPLE 3

### Content Mixing Assay

HeLa and HeLa-CD4⁺ cells were grown to confluency on collagen-coated chamber slides. The cells were then coinfected with Ad5GFP and Ad5HIVenv at a multiplicity of infection (MOI) of 10 plaque-forming units (PFU) per cell for 1 hour at 37° C. Approximately 30 hr later, the monolayers were visualized by light and fluorescence microscopy (see Microscopy, below).

### EXAMPLE 4

### Quantification of Gene Transfer Efficiency

To assess Ad infection efficiency, 10⁵ HeLa or HeLa-CD4⁺ cells were plated into triplicate wells of 12-well plates in the presence of 1 ml of culture medium and were allowed to adhere overnight. The cells were incubated in medium (300 μgl/well) containing 2% fetal bovine serum (FBS) with various MOIs of virus at 37° C. in 5% CO₂ for 1 hr, washed with PBS, pH 7.4, and then 1 ml of complete medium was added. Twenty-four hours after infection, the cells were rinsed with PBS and assayed for luciferase activity and protein concentration.

### EXAMPLE 5

### Quantification of Virus Yield

HeLa and HeLa-CD4⁺ cells were infected with Ad5HIVenv as described above at 10 PFU/cell. After a 1-hr adsorption period, the cell monolayers were washed once with fresh growth medium and cells were incubated with 2 ml of fresh growth medium at 37° C. in a CO₂ incubator. At 24-hr intervals postinfection, the medium (medium fraction) was collected and the cell monolayer (cell fraction) was scraped into 2 ml of fresh medium. Both medium and cell fractions were stored at −70° C. until all time points were collected. The medium and cell fractions were subjected to four freeze-thaw cycles and then centrifuged at 10,000×g for 15 min at room temperature. The amounts of virus present in the resulting supernatants from the medium and cell fractions were then analyzed by standard plaque assays on 293 cells as described previously (Graham and van der Eb, 1973).

### EXAMPLE 6

### Microscopy

Bright-field and fluorescence microscopy of mock- and Ad-infected cells was performed with an Olympus IX 70 inverted epifluorescence microscope equipped with bright-field and fluorescence optics at the UAB Digital Imaging Facility. Image capture and acquisition was performed with IPLab Spectrum software (Scanalytics, Fairfax, Va.). Electron microscopy was performed at the UAB High Resolution Imaging Facility. Samples were prepared by fixation in 3% glutaraldehyde (electron microscopy grade), 0.8% tannic acid in 0.1 M cacodylate buffer for 1 hr followed by rinsing in 0.1 M cacodylate buffer (three times, 5 min each). The monolayer was fixed again in 0.1% osmium tetroxide in 0.1 M cacodylate buffer for 1 hr and then rinsed with 0.1 M cacodylate buffer (three times, 5 min each). Next, the monolayers were dehydrated in sequential washes containing 50, 75, 95, and 100% ethanol (5 min each). The cell monolayers were incubated with Polybed 812 resin and 100% ethanol (1:1) overnight, and then three times for 2 hr in 100% Polybed resin. Finally, the monolayers were embedded into thin layers of 100% Polybed at 60° C. degrees overnight.

### EXAMPLE 7

### Evaluation of Ad-Mediated HIV-1 Env-Induced Syncytium Formation In Hela Cell Lines

The potent antitumor effects of fusogenic membrane glycoproteins suggest their potential use as novel reagents for cancer gene therapy applications (Bateman et al., 2000; Diaz et al., 2000; Fielding et al., 2000; Higuchi et al., 2000). However, the compatibility of fusogenic membrane glycoprotein-induced syncytium formation in the context of an Ad expression system has not been thoroughly evaluated. Therefore, Ad-mediated gene delivery was evaluated in the presence and absence of HIV-1-mediated syncytia.

The present invention develops a novel system consisted of two elements: (1) a replication-competent Ad, Ad5HIVenv, that expresses functional HIV-1 Env, and (2) a matched set of tumor cell lines that are permissive (HeLa-CD4⁺) and nonpermissive (HeLa) for HIV-1 Env-induced syncytium formation. Both cell lines endogenously express the chemokine receptor CXCR4, so that HIV-1 Env-induced syncytia are dependent on the presence of the CD4 receptor.

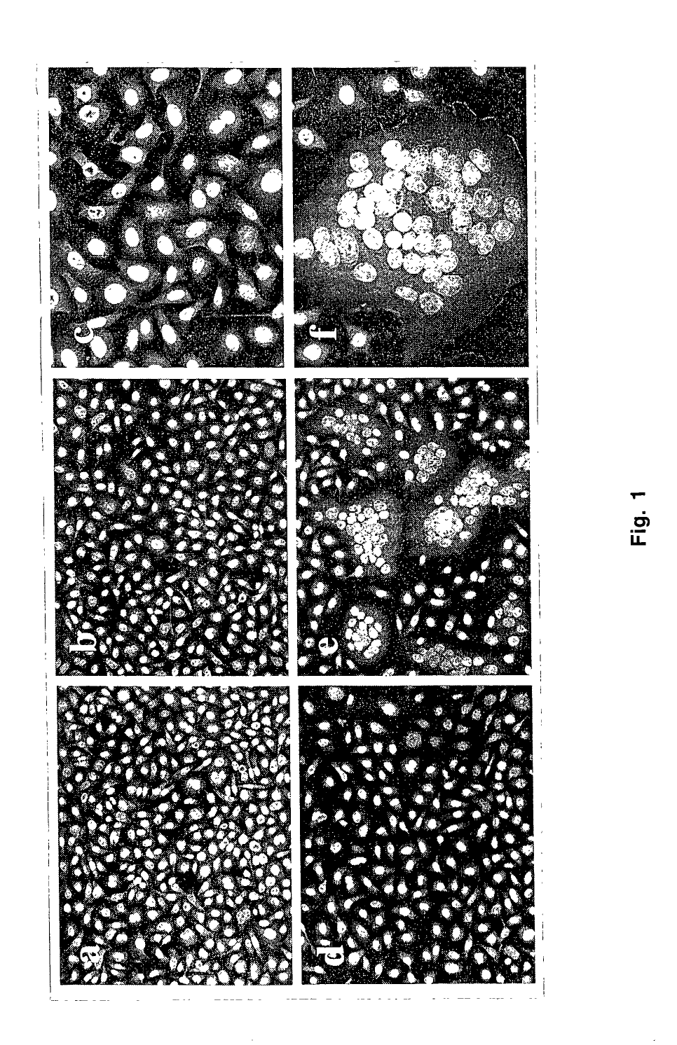

When both cell lines were infected with Ad5HIVenv, syncytia were observed only in HeLa-CD4 1 cells (FIGS. 1e and f). Multiple syncytia composed of 30 to 60 nuclei that involved approximately 50% of the cells in the monolayer were observed 24 hr postinfection (FIGS. 1e and f). No syncytia were observed in the mock-infected HeLa (FIG. 1a) or HeLa-CD4⁺ cells (FIG. 1d) or in the Ad5HIVenv-infected HeLa cells (FIGS. 1b and c). No syncytia were observed in the CD4-negative HeLa cells even when infected at higher MOIs (100 and 1000 PFU/cell) with Ad5HIVenv (data not shown). These data demonstrate the utility and specificity of the fusogenic membrane glycoprotein model for evaluating the effects of syncytium formation in a replicating adenoviral background.

### EXAMPLE 8

### Evaluation of Ad Receptor Expression and Ad-mediated Transgene Expression of HeLa and HeLa CD4⁺ Cells

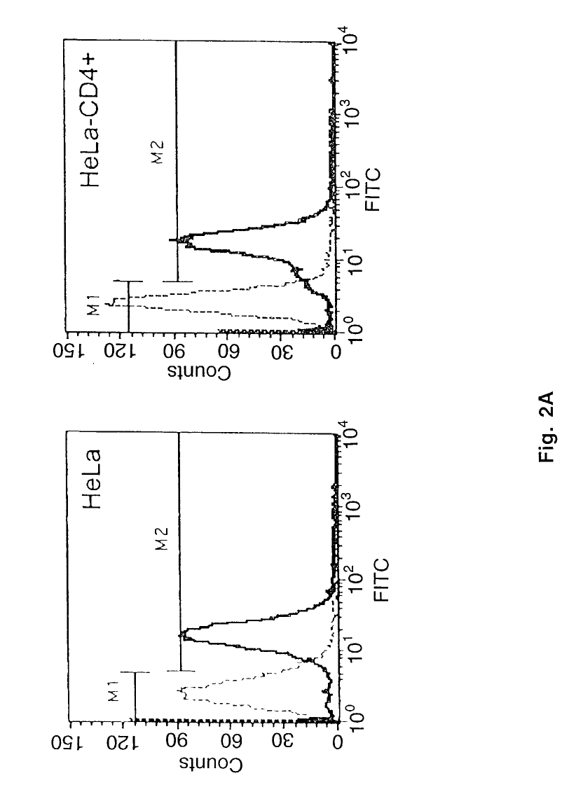

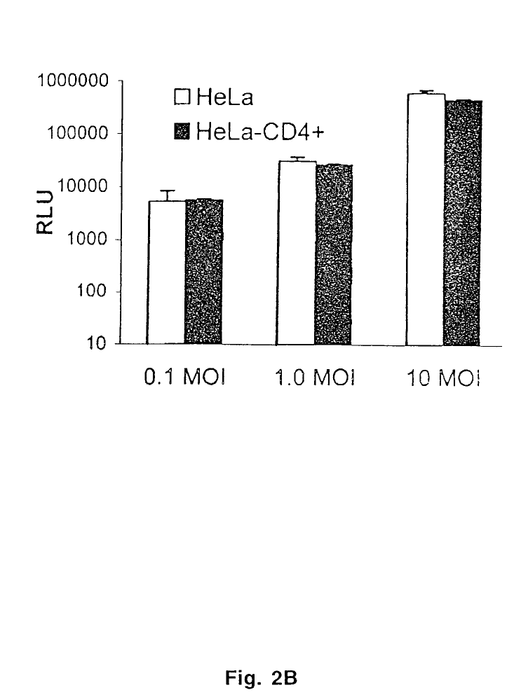

Since it was possible that the modified HeLa-CD4⁺ cells were differentially infected by Ad compared with their parental counterpart (HeLa), they were evaluated for their expression of the primary adenovirus receptor, CAR (FIG. 2A). The HeLa and HeLa-CD4⁺ cells were indistinguishable for CAR expression on the basis of three independent flow cytometry analyses (FIG. 2A). In each analysis, greater than 93% of the HeLa and HeLa-CD4⁺ cells expressed CAR with mean fluorescence intensities between 19.5 and 20.5.

The Ad transduction efficiency of the HeLa and HeLa-CD4⁺ cells was also compared (FIG. 2B). Equivalent numbers of HeLa and HeLa-CD4⁺ cells were infected at MOIs of 0.1, 1.0, and 10 with Ad5Luc1, an Ad that expresses the luciferase reporter gene. At each MOI, the amount of luciferase activity in infected HeLa and HeLa-CD4⁺ cells was not significantly different (p>0.05). Collectively, these data demonstrate that there are no significant differences between HeLa and HeLa-CD4⁺ cells with regard to their Ad transduction or transgene expression efficiencies.

### EXAMPLE 9

### Evaluation of Ad-Mediated Transgene Expression In The Absence and Presence of FMG-Mediated Syncytia

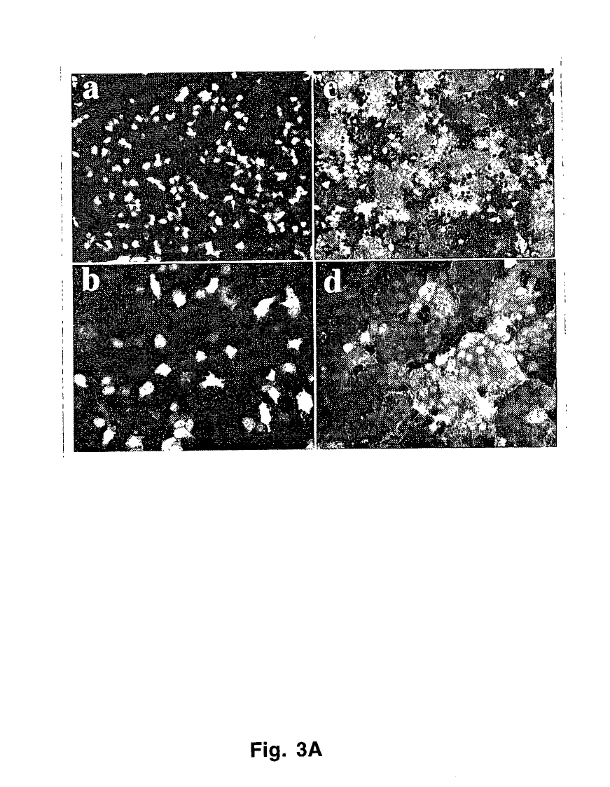

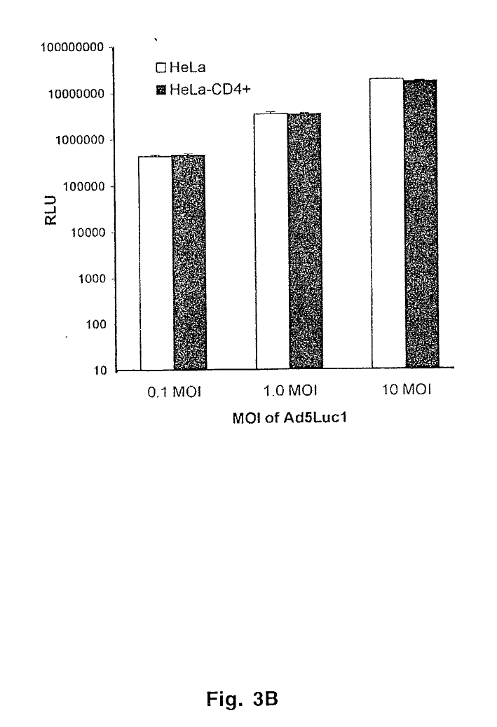

To characterize the pattern of Ad transgene expression in the absence and presence of syncytia, HeLa and HeLa-CD4⁺ cells were coinfected with Ad5HIVenv and Ad5GFP which expresses the green fluorescent protein (GFP). At 24 hr after infection, representative micrographs were recorded (FIG. 3A). In the CD4-negative HeLa cells, individual cells expressed GFP (FIG. 3A, panels a and b); however, in the HeLa-CD4⁺ cells both single cells and syncytial masses expressed GFP (FIG. 3A, panels c and d). These data demonstrate that the Ad transgene product (i.e., GFP) is dispersed throughout the syncytial masses.

Next, the amount of transgene expression in the absence or presence of syncytia was quantified. HeLa and HeLa-CD4⁺ cells were coinfected with Ad5HIVenv and Ad5Luc1. Approximately 24 hours after coinfection, cells were harvested and luciferase activity was measured (FIG. 3B). Luciferase activity was essentially unchanged in the absence (HeLa) and presence (HeLa-CD4⁺) of syncytia, demonstrating that syncytium formation is not deleterious to adenoviral transgene expression. It was noted that luciferase activity was higher in this experiment compared with the lucerifase activity shown in FIG. 2B at equivalent MOIs. The most likely explanation for these data is that Ad5Luc1, a nonreplicating adenovirus, is complemented by the replicating adenovirus AdHIVenv in the experiment shown in FIG. 3B.

However, in the experiment shown in FIG. 2B, only Ad5Luc1 is used. In the former context, Ad5Luc1 DNA is amplified, providing multiple rounds of luciferase expression. In the latter context, Ad5Luc1 DNA is not amplified, therefore only one round of luciferase is expressed. The same increase in luciferase expression was observed under similar coinfection conditions using other replication-competent Ads (data not shown).

### EXAMPLE 10

### Analyses Of FMG-Mediated Ad Particle Production And Dispersion

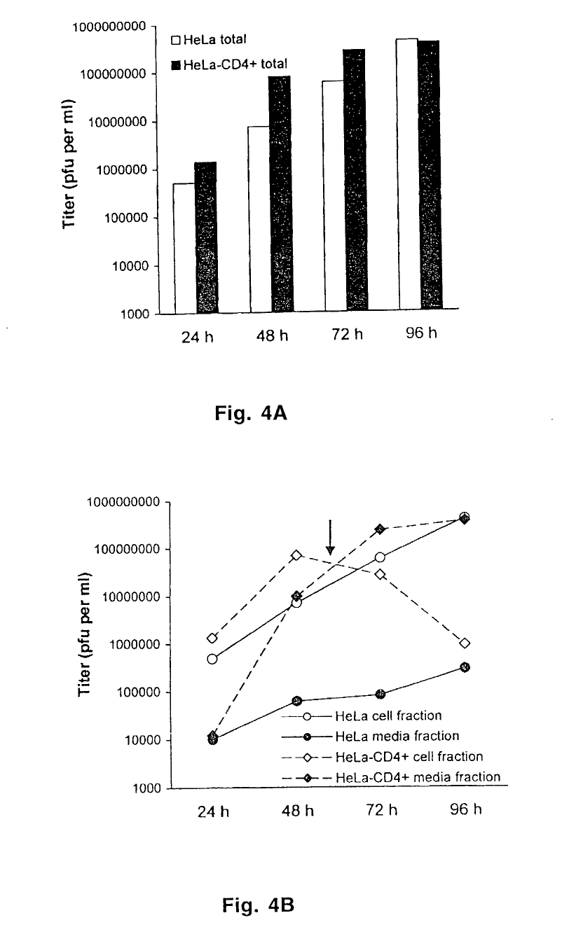

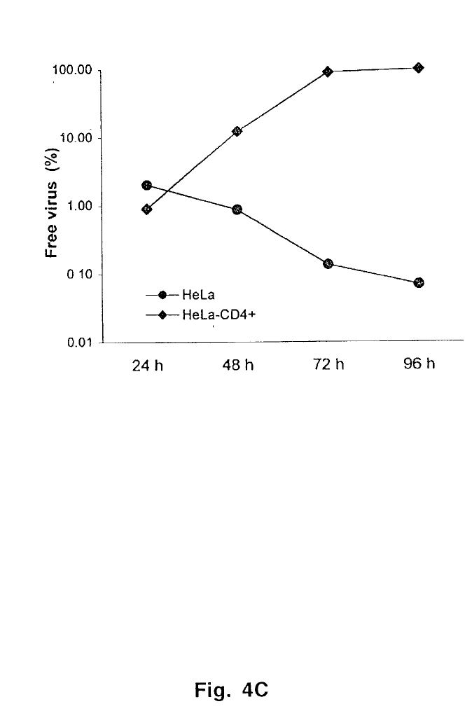

To determine whether syncytium formation is detrimental to de novo Ad particle production, HeLa and HeLa-CD4⁺ cells were infected with Ad5HIVenv and new virus progeny were measured at various time points after infection using plaque assays (FIG. 4A). The amounts of free virus in the medium (medium fraction) and cell-associated virus (cell fraction) were determined separately and then added together to give the net yield of virus at each time point. In the absence of syncytia the HeLa cells yielded a total of 5×10⁵, 7.3×10⁶, 6.1×10⁷, and 4.2×10⁸ PFU/ml 24, 48, 72, and 96 hr after infection respectively. In the presence of syncytia the HeLa-CD4⁺ cells yielded 1.4×10⁶, 8.0×10⁷, 2.7×10⁸, and 3.8×10⁸ PFU/ml at the same time points. The HeLa-CD4⁺ cells produced more virus particles during the first 72 hr; however, by 96 hr after infection both cells produced equivalent amounts of total virus.

Next, the amount of virus in the medium and cell fractions were analyzed separately at the time points listed above (FIG. 4B). In the absence of syncytia, a 3-log increase in cell-associated virus was observed in the HeLa cells and the amount of free virus increased approximately 1 log over the 96-hr infection period. By contrast, the amount of virus associated with the HeLa-CD4⁺ cell fraction increased during the first 48 hr, but then rapidly declined during the next 48-hr time period, and the free virus progressively increased throughout the 96-hr time period in the presence of syncytia. A viral burst occurred between 48 and 72 hr after infection of HeLa CD4⁺ cells that accounted for this rapid increase in the amount of free virus released into the medium.

A graphical representation of the ratio of free to total virus (titer of free virus divided by the sum of the titers of the free and cell-associated virus) in the absence or presence of syncytia is shown in FIG. 4C. At 24 hr after infection, 0.98% of the virus was in the cell fraction of both the HeLa and HeLa-CD4⁺ cells. At each subsequent time point, 5% of the virus was released into the HeLa cell culture medium. However, a different pattern of virus distribution occurred in the HeLa-CD4⁺ cells in the presence of syncytia. At 48, 72, and 96 hr after infection, 12, 90, and 99.7% respectively, of the virus was released into the culture medium of the HeLa-CD4⁺ cells. At the 96-hr time point, more than 99% of the virus had been released into the medium from the HeLa-CD4⁺ cells compared with less than 0.1% of the virus from the HeLa cells, which produced a net difference of free-to-total virus of approximately three orders of magnitude.

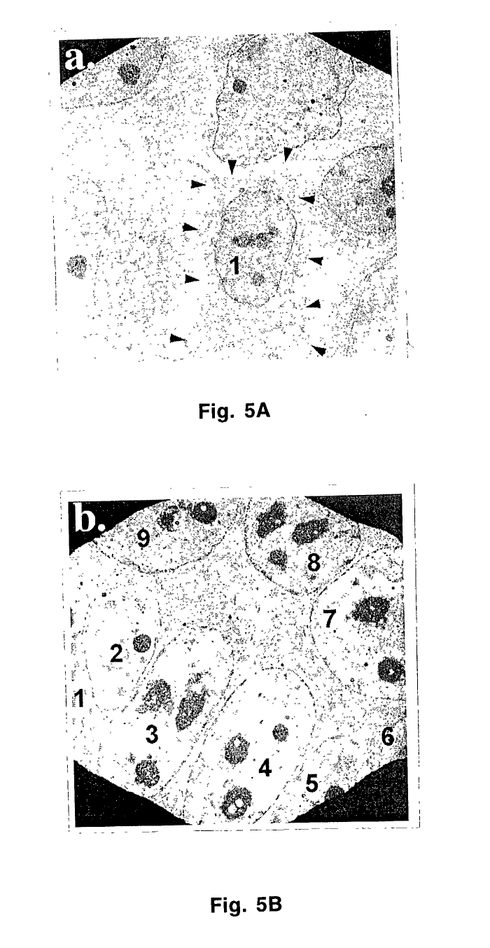

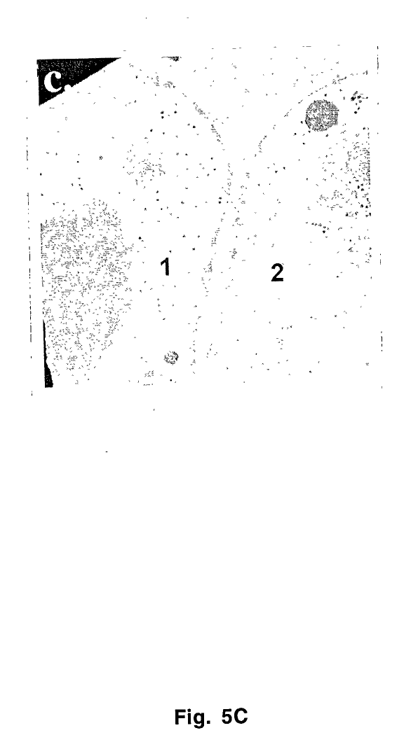

The newly formed adenoviral particles within the syncytial masses were then characterized. HeLa and HeLa-CD4⁺ cells were infected with Ad5HIVenv and then visualized by electron microscopy (FIG. 4). HeLa cells infected with Ad5HIVenv had the expected virus-induced nuclear changes such as chromatin condensation (in the nucleoli and nuclear lamina), amorphously dense regions, granular regions, and crystalline protein arrays (Zhai et al., 1987; Chaly and Chen, 1993; Puvion-Dutilleul et al., 1995, 1999) and displayed clear interstitial boundaries delineating individual cells (FIG. 5a, arrowheads). Individual nuclei of the infected HeLa cells contained adenovirus particles, which represented approximately 8% of the total nuclei examined (n=300; Table 1).

Infected HeLa-CD4⁺ cells displayed virus-induced nuclear changes similar to those observed for infected HeLa cells. However, the majority (0.80%) of the infected HeLa-CD4⁺ cells were involved in syncytia as indicated by the lack of interstitial boundaries between cells (FIG. 5b and Table 1). The nuclei of both HeLa and HeLa-CD4⁺ cell were also examined for the presence of Ad particles, as represented in FIG. 5c. Within the syncytia examined, approximately 80% contained 10-20 nuclei (n=300) that were positive for Ad particles (Table 1). These data demonstrate that the Ad particles redistribute throughout the nuclei in syncytia, resulting in a dramatic increase in the total number of infected cells.

TABLE 1
Summary Of Adenovirus Dispersion In The Presence
And Absence Of Syncytia ^{a}

| CELL: | HeLa | HeLa-CD4+ | HeLa | HeLa-CD4+ |
|---|---|---|---|---|
| VIRUS: | Ad5Luc3 | Ad5Luc3 | Ad5HIVenv | Ad5HIVenv |
| Number of | 200 | 200 | 300 | 300 |
| nuclei counted | | | | |
| Number of | 0 | 0 | 0 | 247 |
| cells in | | | | |
| syncytia ^{b} | | | | |
| Number of | 18 | 15 | 24 | 195 |
| infected cells ^{c} | | | | |
| Infected nuclei | NA | NA | NA | 160 |
| in syncytia ^{d} | | | | |
| Percentage of | 9 | 7.5 | 8 | 65 |
| total nuclei | | | | |
| infected | | | | |

NA, Not applicable.
^{a} Cells were infected with either the control virus Ad5Luc3, or the experimental virus Ad5HIVenv 28 hr before fixing and preparing for electron microscopy analyses.
^{h} Syncytia were defined as regions of multiple nuclei devoid of apparent interstitial boundaries between cells compared with the controls.
^{c} Cells were scored positive for infection by the presence of 10 or more virus particles detected in the nucleus at ×100,000.
^{d} Nuclei were scored positive for infection by the presence of 10 or more virus particles detected at ×100,000.

### EXAMPLE 11

### Syncytial Formation Induced by Mason-Pfizer Monkey Virus Fusogenic Membrane Glycoprotein in Prostate Cancer Cells

Since Mason-Pfizer monkey virus (MPMV) envelope glycoprotein utilizes ubiquitous cell surface receptors for syncytia formation, it is reasonable to expect that it will also induce syncytia formation in a variety of other cancer cell types. As the next step to test the flexability of the present invention, fusogenic membrane glycoprotein from Mason-Pfizer monkey virus was transiently expressed from the plasmid vector, designated pMPMVenv, in the prostate cancer cell line LNCaP to demonstrate the ability to induce syncytia. The LNCaP cells were purchased from the American Tissue Culture Collection and pMPMVenv was obtained from Dr. Eric Hunter.

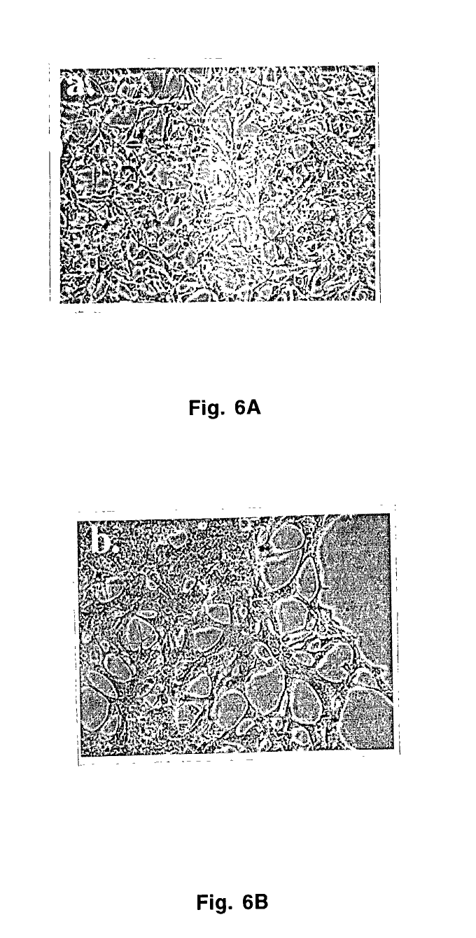

FIG. 6 shows that syncytia was only induced in prostate cancer cells transfected with pMPMVenv that expresses the Mason-Pfizer monkey virus fusogenic membrane glycoprotein, but not in cells transfected with an irrelevant plasmid that expresses the luciferase reporter gene.

### EXAMPLE 12

### Model of Efficient Syncytium-Induced Dispersion of Ad Gene Products

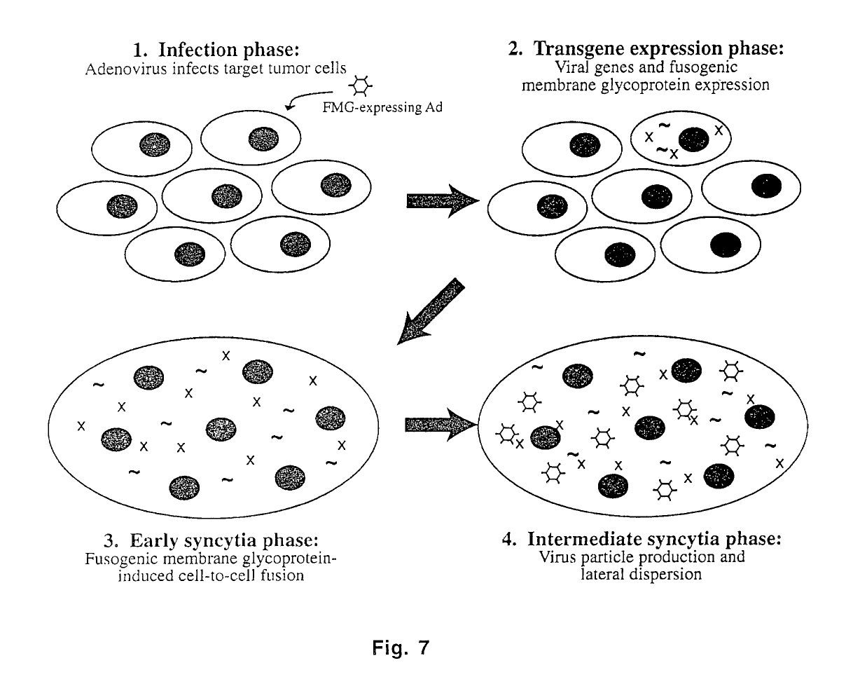

On the basis of the data presented here, a model of efficient syncytium-induced dispersion of Ad gene products and of de novo-synthesized viral particles was proposed (FIG. 7). After infection of the target tumor cells (infection phase), the fusogenic membrane glycoprotein and viral genes are expressed (transgene expression phase). The expressed fusogenic membrane glycoproteins cause nearby cells to fuse with the originally infected cell (early syncytia phase) and, subsequently, the viral gene products are dispersed throughout the syncytial mass. As the adenovirus replication cycle continues (intermediate syncytia phase), virus particles assemble and disperse throughout the syncytial mass. Thus, the pattern of Ad replication within a target tumor mass is altered by fusogenic membrane glycoprotein-mediated syncytium formation through increased dispersion of both the transgene product and de novo-synthesized virus particles from infected to uninfected tumor cells.

Although the data presented in this study support this model, it not clear how progeny virus particles are spread from nucleus to nucleus since it is generally believed that Ad disassembly within an endosome (originating on the cell membrane) is required for nuclear transport. It is likely that fusogenic membrane glycoprotein-induced syncytium formation alters the native biology of Ad replication.

The following references were cited herein:

Alemany et al. (2000). Nat. Biotechnol. 18:723-727.

An et al. (2001). J. Virol. 75:3488-3489.

Bateman et al. (2000). Cancer Res. 60:1492-1497.

Bischoff et al. (1996). Science 274:373-376.

Brody and Hunter. (1992). J. Virol. 66:3466-3475.

Chaly and Chen. (1993). Biochem. Cell Biol. 71:475-487.

Chong et al. (2001). In the Fourth annual meeting of the American Society Of Gene Therapy, Seattle, Wash.

Curiel. (2000). Clin. Cancer Res. 6:3395-3399.

Dachs et al. (1997). Oncol. Res. 9:313-325.

Dewar et al. (1989). J. Virol. 63:129-136.

Diaz et al. (2000). Gene Ther. 7:1656-1663.

Doronin et al. (2001). J. Virol. 75:3314-3324.

Duprex et al. (1999). J. Virol. 73:9568-9575.

Fielding et al. (2000). Hum. Gene ther. 11:817-826.

Firsching et al. (1999). J. Virol. 73:5265-5273.

Fueyo et al. (2000). Oncogene 19:2-12.

Galanis et al. (2001). Hum. Gene Ther. 12:811-821.

Ganly et al. (2000). Clin. Cancer Res. 6:798-806.

Graham and Prevec. (1991). Manipulation of adenovirus vectors. In molecular biology, gene transfer and expression techniques. Murray and Walker, eds. (Humana Press, Clinton, N.J.) pp. 109-128.

Graham and Van Der Eb. (1973). Virology 52:456-467.

Hallenbeck et al. (1999). Hum. Gene Ther. 10:1721-1733.

Haviv et al. (2001). In the 4th Annual American Society Of Gene Therapy, Seattle, Wash.

Higuchi et al. (2000). Cancer Res. 60:6396-6402.

Hoffman and Doms. (1998). AIDS 12:s17-s26.

Hsu et al. (1988). J. Virol. 62:1647-1652.

Huang. (1985). Zhonghua Zhong Liu Za Zhi. 7:332-334.

Hunter. (1997). Viral entry and receptors. In retroviruses. Coffin and Varmus, eds. (Cold Spring Harbor Laboratory Press, Plainview, N.Y.) pp. 71-121.

Jain. (1990a). Cancer Res. 50:814s-819s.

Jain. (1990b). Cancer Metastasis Rev. 9:253-266.

Johnson et al. (2001). In the Fourth Annual Meeting Of The American Society Of Gene Therapy, Seattle, Wash.

Kabat et al. (1994). J. Virol. 68:2570-2577.

Kasono et al. (1999). Clin. Cancer Res. 5:2571-2579.

Kirn. (2000). Oncogene 19:6660-6669.

Krasnykh et al. (1998). J. Virol. 72:1844-1852.

Kuriyama et al. (2000). Hum. Gene Ther. 11:2219-2230.

Kwong et al. (1998). Nature 393:648-659.

Littman. (1998). Cell 93:677-680.

Lorence et al. (1994). Cancer Res. 54:6017-6021.

Luciw. (1996). Fields Virology. (Lippincott-Raven, Philadelphia).

Maizel et al. (1968). Virology 36:126-136.

Melcher et al. (1998). Nat. Med. 4:581-587.

Merzouki et al. (1995). Cell. Mol. Biol. 41:445-452.

Mi et al. (2000). Nature 403:785-789.

Moller et al. (1985). Proc. Natl. Acad. Sci. U.S.A. 82:2456-2459.

Nevo et al. (2001). J. Gene Med. 3:42-50.

Ogura et al. (1978). Arch. Virol. 57:195-198.

Puvion-Dutilleul et al. (1995). Exp. Cell Res. 218:9-16.

Puvion-Dutilleul et al. (1999). Biol. Cell 91:617-628.

Reichard et al. (1992). J. Surg. Res. 52:448-453.

Reynolds et al. (1999). Gene Ther. 6:1336-1339.

Rodriguez et al. (1997). Cancer Res. 57:2559-2563.

Sattentau and Moore. (1993). Philos. Trans. R. Soc. Lond. B Biol. Sci. 342:59-66.

Sauthoff et al. (2000). Hum. Gene Ther. 11:379-388.

Schattner et al. (1985). Cell. Immunol. 90:103-114.

Sinkovics and Horvath. (1993). Intervirology 36:193-214.

Staba et al. (2000). Cancer Gene Ther. 7:13-19.

Suzuki et al. (2001). Clin. Cancer Res. 7:120-126.

Tamura et al. (1997). Science 278:117-120.

Vanderkwaak et al. (1999). Gynecol. Oncol. 74:227-234.

Westendorp et al. (1995). Nature 375:497-500.

Wickham et al. (1997). J. Virol. 71:8221-8229.

Wyss-Coray et al. (1996). J. Clin. Invest. 97:789-798.

Yu et al. (1999a). Cancer Res. 59:4200-4203.

Yu et al. (1999b). Cancer Res. 59:1498-1504.

Yu et al. (2001). Cancer Res. 61:517-525.

Zhai et al. (1987). J. Virol. 61:1007-1018.

Zhang. (1999). Cancer Gene Ther. 6:113-138.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

One skilled in the art will appreciate readily that the present invention is well adapted to carry out the objects and obtain the ends and advantages mentioned, as well as those objects, ends and advantages inherent herein. The present examples, along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A method of increasing adenoviral vector dispersion within a tumor mass, comprising the steps of:
infecting said tumor mass with an adenoviral vector comprising a viral fusogenic membrane glycoprotein; and
inducing syncytia formation in said tumor mass by said fusogenic glycoprotein, wherein said syncytia formation would increase dispersion of said adenoviral vector within said tumor mass.

2. The method of claim 1, wherein said fusogenic membrane glycoprotein is from a virus selected from the group consisting of human immunodeficiency type-1 virus, Mason-Pfizer monkey virus, measles virus, human endogenous retrovirus and respiratory syncytia virus.

3. The method of claim 2, wherein said HIV-1 fusogenic membrane glycoprotein are gp120 and gp41.

4. The method of claim 2, wherein said tumor mass is selected from the group consisting of cervical cancer cells, prostate cancer cells, squamous cell carcinoma of the head and neck, breast cancer and lung cancer.
